(19) United States
(12) Patent Application Publication
(10) Pub. No.: US 2021/0137486 A1
(43) Pub. Date: May 13, 2021

(54) INFORMATION ACQUISITION METHOD USING HEART AND LUNG SOUNDS

(71) Applicants: THE ASAN FOUNDATION, Seoul (KR); UNIVERSITY OF ULSAN FOUNDATION FOR INDUSTRY COOPERATION, Ulsan (KR)

(72) Inventor: Sung Hoon KIM, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 17/153,491

(22) Filed: Jan. 20, 2021

Related U.S. Application Data
(63) Continuation of application No. PCT/KR2019/010389, filed on Aug. 14, 2019.

(30) Foreign Application Priority Data
Aug. 14, 2018 (KR) 10-2018-0094911
Aug. 31, 2018 (KR) 10-2018-0103516
Aug. 31, 2018 (KR) 10-2018-0103566

(51) Int. Cl. A61B 7/04; A61B 7/00; A61B 5/0245; A61B 5/00; A61B 5/28

(52) U.S. Cl. CPC A61B 7/04; A61B 7/003; A61B 5/28; A61B 5/7264; A61B 5/0245

(57) ABSTRACT

An information acquisition method using heart and lung sounds is provided. The information acquisition method includes acquiring, by a computer, heart sound measurement data of a specific patient in real time, acquiring, by the computer, at least one processed heart sound measurement data using the heart sound measurement data in real time, extracting, by the computer, a scale factor between the processed heart sound measurement data and a cardiovascular data value, and calculating and providing, by the computer, a calculated value of specific cardiovascular information data based on the processed heart sound measurement data and the scale factor, wherein the cardiovascular information data has the same variation tendency as a variation tendency of specific processed heart sound measurement data.

### CROSS-REFERENCE TO RELATED APPLICATIONS

The present application is a continuation of International Patent Application No. PCT/KR2019/010389, filed on Aug. 14, 2019, which is based upon and claims the benefit of priority to Korean Patent Application Nos. 10-2018-0094911 filed on Aug. 14, 2018, 10-2018-0103516 filed on Aug. 31, 2018, and 10-2018-0103566 filed on Aug. 31, 2018. The disclosures of the above-listed applications are hereby incorporated by reference herein in their entirety.

### BACKGROUND

Embodiments of the inventive concept described herein relate an information acquisition method using heart and lung sounds.

Heart and lung sounds acquired by auscultation of a heart or lungs may be used as data for diagnosing a patient's disease. However, in a process of acquiring the heart and lung sounds, various signals may be mixed therewith or a signal quality may vary depending on positions at which the heart and lung sounds are measured. Using the heart and lung sounds that contain noise or have poor signal quality may disallow accurately diagnosing the patient's health or disease.

Accordingly, there is a need for a method for acquiring the heart and lung sounds that may be used as accurate analysis data. Further, there is a need for a method for acquiring heart and lung sounds with an accurate signal pattern as pre-process data, and using the same as a variety of analysis indicators.

Cardiovascular information refer to information relating to a heart or a blood vessel, and may include a stroke volume (SV), a systemic vascular resistance (SVR), a pulse pressure (PP), a stroke volume variation (SVV) and a systolic time variation (STV), etc. Pathological conditions in an air duct corresponding to lung information include secretion in the air duct, mucus plug in the air duct, and sputum in the air duct.

In order to acquire the cardiovascular information or the lung information, invasive methods or indirect acquisition methods are mainly used. It may be difficult to implement most of the invasive methods during surgery. The indirect acquisition methods (e.g., ultrasonic measurement method, end-tidal carbon dioxide partial pressure, monitoring of pressure in the air duct, etc.) may not acquire accurate values.

### SUMMARY

Embodiments of the inventive concept are to provide cardiovascular and lung information which should otherwise be acquired invasively, using heart and lung sounds of a patient acquired in real time.

Further, embodiments of the inventive concept are to provide a tendency of increase and decrease of cardiovascular and lung information over time using a non-invasive method.

Further, embodiments of the inventive concept are to provide an accurate value of cardiovascular and lung information using a non-invasive method.

Further, embodiments of the inventive concept are to provide a method and device for acquiring heart and lung sound signals.

The purposes to be solved by the inventive concept are not limited to the purposes mentioned above. Other purposes as not mentioned will be clearly understood by those skilled in the art from following descriptions.

According to an exemplary embodiment, an information acquisition method using heart and lung sounds includes acquiring, by a computer, heart sound measurement data of a specific patient in real time, acquiring, by the computer, at least one processed heart sound measurement data using the heart sound measurement data in real time, extracting, by the computer, a scale factor between the processed heart sound measurement data and a cardiovascular data value, and calculating and providing, by the computer, a calculated value of specific cardiovascular information data based on the processed heart sound measurement data and the scale factor, wherein the cardiovascular information data has the same variation tendency as a variation tendency of specific processed heart sound measurement data.

The method further includes, creating and providing, by the computer, a real-time variation graph of the processed heart sound measurement data, wherein the real-time variation graph of the processed heart sound measurement data has the same variation tendency as a variation tendency of a specific cardiovascular information data graph.

The processed heart sound measurement data includes a second heart sound maximum amplitude or a power, wherein the cardiovascular information data includes a systemic vascular resistance (SVR).

The processed heart sound measurement data includes a systolic time interval (S1-S2 interval, STI), wherein the cardiovascular information data includes a stroke volume (SV), a pulse pressure (PP) or a pulse pressure variation (PPV).

The processed heart sound measurement data includes a first heart sound maximum amplitude or a power, wherein the cardiovascular information data includes a cardiac muscle contractility variation, wherein the cardiac muscle contractility variation is calculated as a peak of a waveform obtained by differentiating an arterial pressure waveform in one heart period.

The acquiring of the heart sound measurement data includes acquiring the heart sound measurement data using heart and lung sound data containing the heart sound measurement data via a heart and lung sounds acquisition device inserted to a specific point in an airway or an esophagus of the patient.

The method further includes deriving, by the computer, a correlation between processed reference heart sound data and cardiovascular invasive measurement data, using the processed reference heart sound data and the cardiovascular invasive measurement data, wherein the processed reference heart sound data and the cardiovascular invasive measurement data are acquired from at least one the same patient and at the same time.

According to an exemplary embodiment, an information acquisition method using heart and lung sounds includes acquiring, by a computer, lung sound measurement data of a specific patient in real time, acquiring, by the computer, at least one processed lung sound measurement data using the processed lung sound measurement data, calculating, by the computer, a scale factor between the lung sound measurement data and a lung information data value, and calculating and providing, by the computer, a calculated value of specific lung information data based on the processed lung sound measurement data and the scale factor.

The method further includes creating, by the computer, a variation graph of the processed lung sound measurement data to provide lung information data.

The processed lung sound measurement data includes frequency region data of the lung sound measurement data, wherein the lung information data includes at least one selected from a group consisting of a lung water amount, secretion in an air duct, mucus plug in the air duct, sputum in the air duct, a pulmonary alveolus opening and closing pressure and pulmonary edema.

The variation graph of the processed lung sound measurement data includes at least one of a power variation based on a frequency band or a combination of frequency band differences, wherein the variation graph of the processed lung sound measurement data has the same variation tendency as a variation tendency of a specific lung information data graph.

Calculating, by the computer, the scale factor between the processed lung sound measurement data and the lung information data value by the computer includes acquiring, by the computer, the scale factor using a graph or a matrix created based on a lung sound and lung invasive data previously acquired from a plurality of patients.

The method further includes, before acquiring, by the computer, the lung sound measurement data of the specific patient in real time, deriving, by the computer, a correlation between the processed reference lung sound data and lung invasive measurement data.

According to an exemplary embodiment, an information acquisition method using heart and lung sounds includes acquiring, by a computer, a heart sound and/or a lung sound based on a measurement position of a probe for measuring a heart sound and/or a lung sound of a patient, analyzing, by the computer, the heart sound and/or the lung sound based on the measurement position and determining, by the computer, a target measurement position of the probe based on the analyzing result, and providing, by the computer, the target measurement position of the probe.

Acquiring, by the computer, the heart sound and/or the lung sound includes determining, by the computer, at least one measurement position of the probe, and acquiring, by the computer, the heart sound and/or the lung sound at each of the at least one determined measurement position.

Determining, by the computer, the target measurement position of the probe includes extracting, by the computer, a signal waveform of the heart sound and/or the lung sound acquired at each of the at least one measurement position, analyzing, by the computer, the signal waveform of the heart sound and/or the lung sound and determining, by the computer, whether the analyzed signal waveform is an appropriate signal waveform, and deriving, by the computer, a measurement position of the probe corresponding to the appropriate signal waveform based on the determination result and determining, by the computer, the derived measurement position as the target measurement position.

Acquiring, by the computer, the heart sound and/or the lung sound includes filtering the heart sound and the lung sound based on a difference between frequency bands of the heart sound and/or the lung sound, and separating the heart sound and the lung sound from each other, or separating the heart sound into a first heart sound and a second heart sound, based on an amplitude or an amplitude variation of a signal waveform of the heart sound and/or the lung sound, or separating the lung sound into a left lung sound and a right lung sound, based on a frequency band of the heart sound and/or the lung sound.

Acquiring, by the computer, the heart sound and/or the lung sound includes acquiring the heart sound and/or the lung sound using a microphone, while a position of the microphone is adjusted based on percentages of the heart sound and lung sound to be acquired.

The method further includes analyzing, by the computer, the heart sound and/or the lung sound and determining, by the computer, whether an abnormal region is contained therein, based on the analysis result, and when the computer determines that the heart sound and/or the lung sound contains the abnormal region, correcting, by the computer, a signal waveform of the abnormal region based on a predefined normal signal waveform.

Determining, by the computer, whether the abnormal region is contained therein includes acquiring, by the computer, training data learned from a plurality of patients, and determining, by the computer, whether the abnormal region is contained therein, based on the training data, wherein the training data is created by collecting, by the computer, the heart sound and/or the lung sound containing an abnormal region from the plurality of patients and performing, by the computer, deep learning on the collected heart sound and/or lung sound.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram for describing a method for providing cardiovascular information using a heart sound as a graph according to one embodiment of the inventive concept;

FIG. 2 is a diagram for describing a method for calculating a calculated value of cardiovascular information using a heart sound and providing the calculated value according to one embodiment of the inventive concept;

FIG. 3 is a diagram for describing a method for deriving a correlation between a reference heart sound data and invasive measurement data according to the inventive concept;

FIGS. 4A and 4B are graphs for describing a relationship between a second heart sound amplitude and a systemic vascular resistance according to one embodiment of the inventive concept;

FIGS. 5A and 5B are graphs for describing a relationship between a systolic time interval and a stroke volume according to one embodiment of the inventive concept;

FIGS. 6A and 6B are graphs for describing a relationship between a systolic time interval and a pulse pressure according to one embodiment of the inventive concept;

FIGS. 7A and 7B are graphs for describing a relationship between a systolic time variation and a pulse pressure variation according to one embodiment of the inventive concept;

FIGS. 8A and 8B are graphs for describing a relationship between a first heart sound and a cardiac muscle contractility according to one embodiment of the inventive concept;

FIG. 9 is a diagram for describing a method for extracting cardiovascular data using a correlation and heart sound measurement data according to the inventive concept;

FIGS. 10 and 11 are diagrams for describing a method for removing noise when deriving a correlation between reference heart sound data and invasive measurement data according to the inventive concept;

FIG. 12 is a diagram for describing a method for creating a matrix for extracting a scale factor according to the inventive concept;

FIG. 13 is a diagram for describing a method for providing lung information using a lung sound as a graph according to one embodiment of the inventive concept;

FIG. 14 is a diagram for describing a method for calculating a calculated value of lung information using a lung sound and providing the calculated value according to one embodiment of the inventive concept;

FIG. 15 is a diagram for describing a method for deriving a correlation between reference lung sound data and invasive measurement data according to the inventive concept;

FIG. 16 is a graph to allow identification of presence or absence of secretion in an air duct based on a power variation based on a lung sound frequency band according to one embodiment of the inventive concept;

FIG. 17 is a diagram for describing a method for extracting lung information data using a correlation and lung sound measurement data according to the inventive concept;

FIG. 18 and FIG. 19 are diagrams for describing a method for removing noise when deriving a correlation between reference lung sound data and invasive measurement data according to the inventive concept;

FIG. 20 is a diagram for describing a method for creating a matrix for extracting a scale factor according to the inventive concept;

FIG. 21 is a flow chart showing a method for acquiring heart and lung sound signals according to one embodiment of the inventive concept;

FIGS. 22A to 22D are diagrams showing a signal waveform of a heart sound and/or a lung sound acquired based on a measurement position of a probe;

FIG. 23 is a flow chart showing a method for acquiring heart and lung sound signals according to another embodiment of the inventive concept;

FIGS. 24 to 26 are diagrams for describing an example of utilizing acquired heart and/or lung sounds according to an embodiment of the inventive concept; and

FIG. 27 is a block diagram showing a device that performs a method for acquiring heart and lung sound signals according to an embodiment of the inventive concept.

### DETAILED DESCRIPTION

Advantages and features of the inventive concept, and a method of achieving them will become apparent with reference to embodiments described below in detail together with the accompanying drawings. However, the inventive concept is not limited to the embodiments disclosed below, but may be implemented in various different forms. The present embodiments are provided to merely complete the disclosure of the inventive concept, and to merely fully inform those skilled in the art of the inventive concept of the scope of the inventive concept. The inventive concept is only defined by the scope of the claims.

The terminology used herein is for the purpose of describing the embodiments only and is not intended to limit the inventive concept. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or greater other features, integers, operations, elements, components, and/or portions thereof. Like reference numerals refer to like elements throughout the disclosure. As used herein, the term “and/or” includes any and all combinations of one or more of the associated listed items. Although terms “first”, “second”, etc. are used to describe various components, it goes without saying that the components are not limited by these terms. These terms are only used to distinguish one component from another component. Therefore, it goes without saying that a first component as mentioned below may be a second component within a technical idea of the inventive concept.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the phrase ‘heart sound measurement data’ refers to heart sound data derived from current heart and lung sound signals of a patient as measured in real time.

As used herein, the phrase ‘processed heart sound measurement data’ refers to processed data as calculated from the heart sound measurement data or some values as derived therefrom. For example, the processed heart sound measurement data includes a first heart sound, a second heart sound, a phonocardiogram (PCG), a systolic time interval S1-S2 interval, STI), a first heart sound maximum amplitude, and a second heart sound maximum amplitude. All data that may be derived by processing the heart sound measurement data are included therein. The present disclosure is not limited thereto.

As used herein, the phrase ‘cardiovascular information data’ refers to information data about heart and blood vessels, that is, current information data about heart and blood vessels of a patient from which heart and lung sound signals are acquired in real time. For example, the information data about the heart and blood vessels includes a stroke volume (SV), a systemic vascular resistance (SVR), a pulse pressure (PP), a pulse pressure variation (PPV), a stroke volume variation (SVV), a systolic time variation (STV) and a cardiac muscle contractility variation. All information data related to the heart and blood vessels are included therein. The present disclosure is not limited thereto.

As used herein, the phrase ‘heart and lung sound data’ refers to data of heart and lung sound signals acquired from a patient's left ventricle.

As used herein, the phrase ‘reference heart sound data’ refers to heart sound data derived from heart and lung sound signals as acquired from a plurality of patients in order to derive a correlation between heart sound data and invasive measurement data.

As used herein, the phrase ‘processed reference heart sound data’ refers to processed data as calculated from the reference heart sound data or some values derived therefrom. For example, the processed reference heart sound data includes a first heart sound (S1), a second heart sound (S2), a phonocardiogram (PCG), a systolic time interval (S1-S2 interval, STI), a first heart sound maximum amplitude, and a second heart sound maximum amplitude. All data that may be derived by processing the reference heart sound data are included therein. The present disclosure is not limited thereto.

The first heart sound is a sound generated when an atrioventricular valve is closed. The second heart sound is a sound generated when an aorta and a lung valve are closed, and indicates end of heart systole and beginning of heart diastole.

As used herein, the phrase ‘cardiovascular invasive measurement data’ refers to data acquired from a plurality of patients previously in order to derive a correlation thereof with heart sound data, and refers to information data about heart and blood vessels of the plurality of patients. For example, the cardiovascular invasive measurement data includes a stroke volume (SV), a systemic vascular resistance (SVR), a pulse pressure (PP), a pulse pressure variation (PPV), a stroke volume variation (SVV), a systolic time variation (STV), and an arterial pressure waveform among data previously acquired from the plurality of patients. All information data related to heart and blood vessels are included therein. The present disclosure is not limited thereto.

As used herein, the phrase ‘cardiovascular reference data’ refers to data for reference in removing noise when deriving a correlation between the reference heart sound data and the cardiovascular invasive measurement data, among data acquired in a non-invasive measurement manner. For example, the cardiovascular reference data includes an electrocardiogram and an arterial blood pressure (ABP). All cardiovascular reference data that may serve as a reference are included therein. The present disclosure is not limited thereto.

As used herein, the phrase ‘lung sound measurement data’ refers to lung sound data derived from current heart and lung sound signals of a patient measured in real time.

As used herein, the phrase ‘processed lung sound measurement data’ refers to processed data as calculated from the lung sound measurement data or some values derived therefrom. For example, data obtained by converting the lung sound measurement data into a frequency region are included therein. All data that may be derived by processing the lung sound measurement data are included therein. The present disclosure is not limited thereto.

As used herein, the phrase ‘lung information data’ refers to information data about a lung, that is, current information data about a lung of a patient from which the heart and lung sound signals are acquired in real time. For example, the lung information data includes a lung water amount, secretion in an air duct, mucus plug in the air duct, sputum in the air duct, a pulmonary alveolus opening and closing pressure and pulmonary edema. All lung-related information data is included therein. The present disclosure is not limited thereto.

As used herein, the phrase ‘reference lung sound data’ refers to lung sound data derived from heart and lung sound signals acquired from a plurality of patients in order to derive a correlation between lung sound data and invasive measurement data.

As used herein, the phrase ‘processed reference lung sound data’ refers to processed data as calculated from the reference lung sound data, or some values derived therefrom. For example, data obtained by converting the lung sound measurement data into a frequency region are included therein. All data that may be derived by processing the lung sound measurement data are included therein. The present disclosure is not limited thereto.

As used herein, the phrase ‘lung invasive measurement data’ refers to data previously acquired from a plurality of patients in order to derive a correlation thereof with the lung sound data, that is, refers to lung information data of the plurality of patients. For example, the lung invasive measurement data includes lung information data such as a lung water amount, secretion in the air duct, mucus plug in the air duct, sputum in the air duct, a pulmonary alveolus opening and closing pressure, and pulmonary edema. All lung-related information data is included therein. The present disclosure is not limited thereto.

As used herein, the phrase ‘reference lung data’ refers to data for reference in removing noise when deriving a correlation between the reference lung sound data and the lung invasive measurement data, among data acquired in a non-invasive measurement manner. For example, the reference lung data includes lung data that may act as a reference. The present disclosure is not limited thereto.

The inventive concept provides a method for acquiring an accurate value of cardiovascular information in real time based on an acquired heart sound faster than when using invasive direct acquisition of cardiovascular information, and a method for acquiring an accurate value of lung information in real time based on an acquired lung sound faster than when directly acquiring lung information invasively.

Further, the inventive concept provides a method in which the heart sound and the lung sound are acquired at the same time, and the heart sound and the lung sound for the acquisition of the cardiovascular information or the lung information are acquired from the patient using a probe.

FIGS. 1 to 12 to be described later describe a method for acquiring cardiovascular information using a heart sound. FIGS. 13 to 20 describe a method for acquiring lung information using a lung sound. FIGS. 21 to 27 describe a method for acquiring heart and lung sound signals.

Hereinafter, an embodiment of the inventive concept will be described in detail with reference to the accompanying drawings.

First, based on FIGS. 1 to 11, a method for acquiring cardiovascular information using a heart sound is described.

FIG. 1 is a diagram for describing a method for providing cardiovascular information using a heart sound as a graph according to one embodiment of the inventive concept.

Referring to FIG. 1, a cardiovascular information acquisition method using a heart sound according to one embodiment of the inventive concept includes step S110 of real-time acquisition of heart sound measurement data of a specific patient, step S130 of acquiring processed heart sound measurement data using the heart sound measurement data and step S150 of creating and providing a real-time variation graph of the processed heart sound measurement data.

Step S110 of real-time acquisition of the specific patient's heart sound measurement data acquires the heart sound measurement data using heart and lung sound data including the heart sound measurement data via a heart and lung sounds acquisition device inserted to a specific point of the patient's airway.

The heart and lung sounds acquisition device is configured to include a tube, a probe, a microphone and a connector. The tube is formed to extend from the heart and lung sounds measurement position in an esophagus or an air duct to an outside of the body. The probe is disposed at a first end of the tube and is placed in the heart and lung sounds measurement position to collect heart and lung sounds through the esophagus or air duct surface. The microphone converts the heart and lung sounds collected from the probe into electrical signals, and is placed inside the probe. The connector is connected to a cable at a second end of the tube, and transmits the electrical signals corresponding to the heart and lung sounds to a cardiovascular information output device.

In step S130 of acquiring the processed heart sound measurement data using the heart sound measurement data, at least one data included in the processed heart sound measurement data is acquired.

In step S150 of creating and providing the real-time variation graph of the processed heart sound measurement data, the real-time variation graph of the processed heart sound measurement data refers to a graph of the processed heart sound measurement data, and has the same variation tendency as that of a specific cardiovascular information data graph.

Conventionally, invasive measurement data may be obtained only when a tool is invasively inserted (e.g., a tool is inserted to the heart to determine a stroke volume). Thus, the variation in data could not be identified in surgery where it is difficult to invasively insert the tool.

According to the inventive concept, even without directly providing a cardiovascular information data graph, providing only the real-time variation graph of the processed heart sound measurement data may allow providing the same variation tendency of the cardiovascular information data to a patient, a doctor, or a user who needs cardiovascular information.

For example, in event of a decrease in a blood pressure, the decrease in the blood pressure occurs due to a decrease in a total load (total blood volume), a decrease in a cardiac muscle contractility, or a systemic vascular resistance. Thus, when a patient's blood pressure drop occurs during surgery of a medical staff, it is necessary to determine the cause of the blood pressure drop to cope with the drop.

In this connection, in order to measure the systemic vascular resistance, as described above, a tool must be inserted invasively. However, in most of the surgery, it may be difficult to invasively insert the tool.

Therefore, according to the inventive concept, a second heart sound maximum amplitude graph whose a tendency is consistent with that of a systemic vascular resistance graph, and which is acquired by processing the heart sound measurement data may be provided. Thus, when a value on the second heart sound maximum amplitude graph decreases, the medical staff recognizes that the systemic vascular resistance decreases, so that the staff may appropriately cope with the decrease in the blood pressure of the patient.

FIG. 2 is a diagram for describing a method for calculating a calculated value of cardiovascular information using a heart sound and providing the calculated value according to one embodiment of the inventive concept.

Referring to FIG. 2, the method for calculating the calculated value of cardiovascular information and providing the calculated value according to one embodiment of the inventive concept includes step S210 of real-time acquisition of heart sound measurement data of a specific patient, step S230 of acquiring processed heart sound measurement data using the heart sound measurement data, step S250 of calculating a scale factor between the processed heart sound measurement data and a cardiovascular data value, and step S270 of calculating the calculated value of the cardiovascular information data based on the processed heart sound measurement data and the scale factor, and providing the calculated value.

The step S210 of acquiring the specific patient's heart sound measurement data in real time and step S230 of acquiring the processed heart sound measurement data using the heart sound measurement data are the same as described above in FIG. 1.

In step S250 of acquiring the scale factor between the processed heart sound measurement data and the cardiovascular information data value, the scale factor for calculating the cardiovascular information data value is acquired using the processed heart sound measurement data. The scale factor is acquired using a graph or a matrix created based on heart sounds and cardiovascular invasive measurement data of a plurality of patients as previously acquired. A specific method for acquiring the scale factor using the graph or the matrix will be described later.

In one embodiment, step S270 of calculating the calculated value of cardiovascular information data based on the processed heart sound measurement data and the scale factor and providing the calculated value may include creating a graph of cardiovascular information data based on the processed heart sound measurement data and the scale factor, and calculating and providing an exact value at each point on the graph as the calculated value of the cardiovascular information data. In another embodiment, without creating the graph of cardiovascular information data, an exact value of cardiovascular information data may be calculated and provided.

FIG. 3 is a diagram for describing a method for deriving a correlation between processed reference heart sound data and cardiovascular invasive measurement data according to the inventive concept.

Referring to FIG. 3, the cardiovascular information acquisition method using a heart sound according to one embodiment of the inventive concept include step S100 of deriving a correlation between processed reference heart sound data and cardiovascular invasive measurement data, step S110 of real-time acquisition of heart sound measurement data of a specific patient, step S130 of acquisition of processed heart sound measurement data using the heart sound measurement data, and step S150 of creating and providing a real-time variation graph of the processed heart sound measurement data.

The step S100 of deriving the correlation between the processed reference heart sound data and the cardiovascular invasive measurement data occurs before the acquisition of the patient's heart sound measurement data measured in real time. In the step 100, the correlation is derived using the processed reference heart sound data and cardiovascular invasive measurement data as previously acquired from a plurality of other patients.

The reference heart sound data and the cardiovascular invasive measurement data may be obtained from at least one the same patient among the plurality of other patients and at the same time and may constitute a pair. The correlation between the processed reference heart sound data obtained by processing the reference heart sound data and the cardiovascular invasive measurement data is derived.

Further, in FIG. 3, it is shown that step S100 of deriving the correlation between the processed reference heart sound data and the cardiovascular invasive measurement data occurs before step S110 of acquiring the heart sound measurement data of a specific patient in real time, step S130 of acquiring the processed heart sound measurement data using the heart sound measurement data, and step S150 of creating and providing the real-time variation graph of the processed heart sound measurement data. Further, step S100 of deriving the correlation between the reference heart sound data and the cardiovascular invasive measurement data may occur before step S210 of real-time acquisition of the specific patient's heart sound measurement data, step S230 of acquiring the processed heart sound measurement data using the heart sound measurement data, step S250 of calculating the scale factor between the processed heart sound measurement data and the cardiovascular data value, and step S270 of calculating the calculated value of the cardiovascular information data based on the processed heart sound measurement data and the scale factor, and providing the calculated value in FIG. 2.

Hereinafter, a method for acquiring cardiovascular information using the heart sound according to the inventive concept is described based on a graph showing the correlation as the embodiment of the correlation between the processed heart sound measurement data and the cardiovascular information data.

FIGS. 4A and 4B are graphs for describing a relationship between a second heart sound amplitude and a systemic vascular resistance according to one embodiment of the inventive concept.

FIG. 4A and FIG. 4B are exemplary graphs for patient 1 and patient 2, respectively.

FIGS. 5A and 5B are graphs for describing a relationship between a systolic time interval and a stroke volume according to one embodiment of the inventive concept.

FIG. 5A and FIG. 5B are exemplary graphs for patient 1 and patient 2, respectively.

FIGS. 6A and 6B are graphs for describing a relationship between a systolic time interval and a pulse pressure according to one embodiment of the inventive concept.

FIG. 6A is a graph for 20 seconds. FIG. 6B is a graph for several hours.

FIGS. 7A and 7B are graphs for describing a relationship between a systolic time variation and a pulse pressure variation according to one embodiment of the inventive concept.

FIG. 7A and FIG. 7B are exemplary graphs for patient 1 and patient 2, respectively.

FIGS. 8A and 8B are graphs for describing a relationship between a first heart sound and a cardiac muscle contractility according to one embodiment of the inventive concept.

FIG. 8A is a graph for 7 hours. FIG. 8B is a graph for 11 hours. FIGS. 8A and 8B are graphs showing a relationship between the variation of the cardiac muscle contractility and the first heart sound, wherein the variation of the cardiac muscle contractility that continuously varies during surgery, as measured based on the first heart sound and an invasive arterial pressure is calculated as a peak of a waveform obtained by differentiating an arterial pressure waveform in one heart sound cycle.

Referring to FIGS. 4A and 4B, it may be identified that a tendency of a graph of a second heart sound amplitude 11 and that of a systemic vascular resistance 13 are similar to each other.

Various factors contribute to generation of the second heart sound. However, the generation of the second heart sound is mainly related to closure of an aortic valve. The closure of the aortic valve is affected by increase or decrease of an afterload.

Therefore, it may be identified that graph forms of the second heart sound amplitude and the systemic vascular resistance are similar to each other via adjusting of the scale value, as shown in FIGS. 4A and 4B.

As described above, deriving the second heart sound amplitude data as the processed heart sound data using the heart sound measurement data, and providing only the second heart sound amplitude data graph may allow the tendency of the systemic vascular resistance graph to be identified.

The tendency may be recognized such that when the second heart sound amplitude 11 increases, the systemic vascular resistance 13 increases, while when the second heart sound amplitude 11 decreases, the systemic vascular resistance 13 decreases.

Therefore, in a situation where it is difficult to acquire the systemic vascular resistance invasively during surgery, only the heart and lung sound data may be acquired without using the invasive acquisition, and increase/decrease tendency of the systemic vascular resistance may be identified based on the heart and lung sound data and may be provided to the medical staff who needs the increase/decrease tendency of the systemic vascular resistance.

Referring to FIGS. 5A and 5B, it may be identified that the tendencies of the graphs of the systolic time interval (S1-S2 interval, STI) 15 and a stroke volume 17 are similar to each other.

The systolic time approximately depends on a duration from a time immediately after closure of the atrioventricular valve to a time of the closure of the aortic valve. This is because the systolic time is affected by an excess or a lack of a preload which the ventricle should eject at a corresponding time.

Therefore, it may be identified that the graph forms of the systolic time interval 15 and the stroke volume 17 are similar to each other via adjusting of the scale value, as shown in FIGS. 5A and 5B.

As described above, deriving systolic time interval data as processed heart sound data using the heart sound measurement data, and providing only the systolic time interval data graph may allow grasping the tendency of the stroke volume graph. Further, when the stroke volume graph is corrected using information such as a heart rate or an electrocardiogram and a time interval of the heart sound, more accurate stroke volume information may be calculated.

As described above in FIGS. 4A and 4B, in a situation where it is difficult to acquire the stroke volume invasively during surgery, only the systolic time interval data may be acquired without the invasive acquisition and may be processed to calculate an index and then increase/decrease tendency of stroke volume based on the index may be supplied to the medical staff who need to grasp the increase/decrease tendency of the stroke volume.

Referring to FIGS. 6A and 6B, it may be identified that the tendencies of the graphs of the systolic time interval 15 and a pulse pressure 19 are similar to each other.

The pulse pressure represents a difference between a systolic blood pressure and a diastolic blood pressure, and reflects delivery of the blood ejected by a cardiac cycle to a periphery in a pressure delivering manner. It is known that the pulse pressure and the stroke volume have a proportional relationship with each other. Therefore, when the variation of the pulse pressure according to breathing is tracked over a systolic time interval, the variation of the pulse pressure according to breathing may also be tracked over a systolic time interval variation.

Therefore, it may be identified that the graph forms of the systolic time interval 15 and pulse pressure 19 are similar to each other via adjusting of the scale value, as shown in FIGS. 6A and 6B.

As described above, deriving the systolic time interval data as the processed heart sound data using the heart sound measurement data, and providing only the systolic time interval data and the graph obtained by processing the same may allow the tendency of the pulse pressure graph to be identified.

As described above in FIGS. 4A and 4B, in a situation where it is difficult to acquire the pulse pressure invasively during surgery, only systolic time interval data may be acquired while not being acquired invasively and the increase/decrease tendency of the pulse pressure based on the systolic time interval data may be provided to the medical staff who needs to grasp the increase/decrease tendency of the pulse pressure.

Referring to FIGS. 7A and 7B, it may be identified that the tendencies of the graphs of a systolic time variation 21 and a pulse pressure variation 23 are similar to each other.

As described above in FIGS. 6A and 6B, when the variation of the pulse pressure according to the breathing is tracked over the systolic time interval, the variation of the pulse pressure according to the breathing may also be tracked over the systolic time interval variation.

Therefore, it may be identified that the graph forms of the systolic time variation 21 and the pulse pressure variation 23 are similar to each other via adjusting of the scale value, as shown in FIGS. 7A and 7B.

As described above, deriving systolic time variation data as the processed heart sound data using the heart sound measurement data, and providing only the systolic time variation data and the graph obtained by processing the same may allow the tendency of the pulse pressure variation graph to be identified.

Referring to FIGS. 8A and 8B, it may be identified that the tendencies of the graphs of a cardiac muscle contractility variation 25 and a first heart sound 27 are similar to each other.

Specifically, when a peak of the waveform obtained by differentiating the arterial pressure waveform in one heart sound period is referred to as a maximum value (dP/dt max) of the arterial pressure waveform variation, FIGS. 8A and 8B are graphs showing a relationship between the first heart sound and the maximum value (dP/dt max) of the arterial pressure waveform variation. The maximum value (dP/dt max) of the arterial pressure waveform variation may be calculated as an indicator of the cardiac muscle contractility variation 25 which continuously varies during surgery. Thus, based on FIGS. 8A and 8B, it may be identified that the tendencies of the graphs of the first heart sound 27 and the maximum value are similar to each other.

The maximum value (dP/dt max) of the arterial pressure waveform variation is also used as a measure for evaluating cardiac muscle contractility variation 25 after administration of dobutamine.

A reason why the tendencies of the graphs of the cardiac muscle contractility variation 25 and the first heart sound 27 are similar to each other as shown in the graph of FIGS. 8A and 8B are that the cardiac muscle contractility refers to a force by which the heart contracts to eject the blood, and the force is transmitted to a mitral valve at an initial contraction. Specifically, the reason is that the first heart sound is composed of a sound of the closure of the mitral valve and the left ventricle and a sound created while the blood passes through a left ventricular outflow tract (LVOT), and thus a physiological effect by the cardiac muscle contraction affects the first heart sound.

Further, as identified in FIGS. 4A to 8B, an extraction time of each of the second heart sound amplitude, the systolic time interval, the systolic time variation data, and the first heart sound as the processed data value is shorter than that of each of the systemic vascular resistance, the stroke volume, the pulse pressure, the pulse pressure variation and the maximum value of the arterial pressure waveform variation as invasively acquired.

The reason is that, when invasively extracting the systemic vascular resistance, the stroke volume, and the pulse pressure, heat should be applied, and dilution occurs in the body, and measurement is possible only after the blood circulates one time around the body, and thus the extraction time takes at least about 5 minutes.

However, when calculating the tendency of the graph or the calculated value of the cardiovascular information data using the processed reference heart sound data indirectly, there is no process of circulating the blood one time around the body for the invasive extraction. Thus, the cardiovascular information data may be acquired in real time and in a shorter time than when acquiring cardiovascular information data invasively.

FIG. 9 is a diagram for describing a method for extracting cardiovascular data using a correlation and heart sound measurement data according to the inventive concept.

Referring to FIG. 9, the cardiovascular information acquisition method using the heart sound according to the inventive concept includes deriving a correlation between processed reference heart sound data and cardiovascular invasive measurement data S100, real-time acquisition of heart sound measurement data of a specific patient S110, acquiring processed heart sound measurement data using the heart sound measurement data S130, creating and providing a real-time variation graph of the processed heart sound measurement data S150, and extracting cardiovascular information data using the correlation and the heart sound measurement data S170.

In one embodiment, in step S170 of extracting the cardiovascular information data using the correlation and the heart sound measurement data, the correlation is derived using a graph.

In another embodiment, extracting the cardiovascular information data may be performed using a learning algorithm based on the reference heart sound data and the cardiovascular invasive measurement data.

As described above in FIG. 3, in FIG. 9, it is shown that step S170 of extracting the cardiovascular information data using the correlation and the heart sound measurement data occurs after step S100 of deriving the correlation between the processed reference heart sound data and the cardiovascular invasive measurement data to step S150 of creating and providing the real-time variation graph of the processed heart sound measurement data. In another embodiment, step S170 of extracting the cardiovascular information data using the correlation and the heart sound measurement data may occur after step S100 of deriving the correlation between the processed reference heart sound data and the cardiovascular invasive measurement data, and step S210 of the real-time acquisition of the specific patient's heart sound measurement data to step S270 of calculating the calculated value of the cardiovascular information data based on the processed heart sound measurement data and the scale factor and providing the calculated value.

FIGS. 10 and 11 are diagrams for describing a method for removing noise in deriving a correlation between processed reference heart sound data and cardiovascular invasive measurement data according to the inventive concept.

The step of deriving the correlation as described above in FIG. 3 may further include a step of removing noise.

Referring to FIG. 10, the step of removing the noise may include acquiring cardiovascular reference data S310, acquiring a feature of reference heart sound data using a heart sound filter S330, matching the cardiovascular reference data with the reference heart sound data from which the feature is acquired S350, and upon determination that an abnormal region is included in a wave of the cardiovascular reference data, excluding reference heart sound data matching the abnormal region as noise S370.

The cardiovascular reference data may refer to data for reference in removing the noise when deriving the correlation between the reference heart sound data and the cardiovascular invasive measurement data, among data acquired in the non-invasive measurement manner. For example, the cardiovascular reference data may include an electrocardiogram, an arterial blood pressure, and the like.

The cardiovascular reference data may be used to identify the abnormal region in real time. The abnormal region refers to a region in which noise is created in the heart and lung sound data due to the patient's movement and posture change during surgery. Because it is difficult to determine the abnormal region using only the reference heart sound data, the abnormal region may be identified using the cardiovascular reference data, and a portion of the reference heart sound data matching the abnormal region may be excluded, thereby to achieve an effect of accurately deriving the correlation between the processed reference heart sound data and the cardiovascular invasive measurement data.

The step S330 of acquiring the feature of the reference heart sound data using the heart sound filter may include removing intermittent noise and separating a breathing sound, and clearly identifying and acquiring the feature of the reference heart sound data necessary for analysis.

The heart sound filter includes any filter for clearly distinguishing the feature of the heart sound.

Further, referring to FIG. 11, the step of removing the noise includes step S410 of converting a graph on a time region of the correlation into a graph on a frequency region, and upon determination that the abnormal region is included in the graph on the frequency region, step S430 of excluding a portion of the time region matching the abnormal region as noise.

In determining whether the abnormal region is included in the graph on the frequency region, when a value of a specific region exceeds a range of a predetermined value in the graph on the frequency region, the specific region is determined as the abnormal region.

Because it is difficult to determine the abnormal region in the graph on the time region, the abnormal region may be identified in the graph on the frequency region, and a portion of the time region matching the abnormal region may be excluded, thereby to achieve an effect of accurately deriving the correlation between the processed reference heart sound data and the cardiovascular invasive measurement data.

FIG. 12 is a diagram for describing a method for creating a matrix for extracting a scale factor according to the inventive concept.

A front step of the method for calculating the calculated value of the cardiovascular information using the heart sound and providing the calculated value, as described above in FIG. 2 may further include a step of creating a matrix for calculating a scale factor using reference heart sound data, and cardiovascular invasive measurement data as previously acquired from a plurality of patients.

Referring to FIG. 12, the step for creating the matrix for extracting the scale factor includes acquiring reference heart sound data, and acquiring processed reference heart sound data using the reference heart sound data S510, acquiring cardiovascular invasive measurement data S530, calculating a scale factor between the processed reference heart sound data and the cardiovascular invasive measurement data S550, and creating a matrix using the processed reference heart sound data, the cardiovascular invasive measurement data and the scale factor S570.

In step S510 of acquiring the reference heart sound data and acquiring the processed reference heart sound data using the reference heart sound data, the reference heart sound data refers to heart sound data previously acquired from a plurality of patients, and the processed reference heart sound data refers to processed data as calculated from the reference heart sound data or some values derived therefrom.

The reference heart sound data and the cardiovascular invasive measurement data may be obtained from at least one the same patient among the plurality of other patients and at the same time and may constitute a pair. The scale factor between the processed reference heart sound data obtained by processing the reference heart sound data and the cardiovascular invasive measurement data is derived.

It may be identified in FIGS. 4A to 6B that the graph tendencies of the processed reference heart sound data and the cardiovascular invasive measurement data are consistent with each other.

Accordingly, in step S550 of calculating the scale factor between the processed reference heart sound data and the cardiovascular invasive measurement data, the scale factor between the processed reference heart sound data and the cardiovascular invasive measurement data as obtained from at least one the same patient among the plurality of other patients and at the same time may be calculated and acquired.

Step S570 of creating the matrix using the processed reference heart sound data, the cardiovascular invasive measurement data and the scale factor may include creating the matrix using the processed reference heart sound data, the cardiovascular invasive measurement data, and the scale factor for a physical condition of each of a plurality of patients.

Creating the matrix using the processed reference heart sound data, the cardiovascular invasive measurement data and the scale factor for the physical condition of each of the plurality of patients may allow a scale factor that matches the patient's body condition and heart sound data on the matrix to be acquired in subsequently calculating the cardiovascular information data value using only the patient's heart sound measurement data. Further, in this case, not only the scale factor may be acquired, but cardiovascular invasive measurement data may also be acquired.

Therefore, a front step of the method for calculating the calculated value of cardiovascular information using the heart sound and providing the calculated value, as described above in FIG. 2 may further include the step of creating the matrix for calculating the scale factor using reference heart sound data, and cardiovascular invasive measurement data as previously acquired from a plurality of patients. Acquiring the scale factor between the processed heart sound measurement data and the cardiovascular data value S250 may include acquiring the scale factor using the created matrix.

Thereafter, in step S270 of calculating the calculated value of cardiovascular information data based on the processed heart sound measurement data and the scale factor and providing the calculated value, the reference heart sound data on the matrix may be compared with initial heart sound measurement data acquired from the patient and the calculated value may be accurately calculated based on the comparison result.

In one embodiment, when acquiring a certain portion of the heart sound measurement data, and acquiring only one accurate calculated value of the cardiovascular information data matching the body condition of the patient matching a portion of the reference heart sound data matching the certain portion, all of the calculated values of cardiovascular information data may be continuously acquired.

Further, calculating the scale factor between the processed reference heart sound data and the cardiovascular invasive measurement data S550 may be performed using a learning algorithm. The step S570 of creating the matrix using the processed reference heart sound data, the cardiovascular invasive measurement data, and the scale factor may be performed using a learning algorithm, thereby providing the matrix using the scale factor to a user.

A cardiovascular information acquisition device using a heart sound according to another embodiment of the inventive concept includes a heart sound measurement data acquisition unit that acquires real-time heart sound measurement data of a specific patient, a processed heart sound measurement data acquisition unit for acquiring at least one processed heart sound measurement data using the heart sound measurement data, and a processed heart sound measurement data variation graph providing unit to create and provide a real-time variation graph of the processed heart sound measurement data. The real-time variation graph of the processed heart sound measurement data has the same variation tendency as that of the specific cardiovascular information data graph.

A cardiovascular information acquisition device using a heart sound according to another embodiment of the inventive concept includes a heart sound measurement data acquisition unit for real-time acquisition of heart sound measurement data of a specific patient; a processed heart sound measurement data acquisition unit for acquiring at least one processed heart sound measurement data in real time using the heart sound measurement data, a scale factor calculating unit for calculating a scale factor between the processed heart sound measurement data and the cardiovascular data value, and a cardiovascular information data calculating unit for calculating and providing the calculated value of the specific cardiovascular information data based on the processed heart sound measurement data and the scale factor, wherein the cardiovascular information data has the same variation tendency as that of the specific processed heart sound measurement data.

The cardiovascular information acquisition device according to the inventive concept has the same configuration as that of the cardiovascular information acquisition method described in FIGS. 1 to 12.

In FIGS. 13 to 20, a method for acquiring lung information using a lung sound is described.

FIG. 13 is a diagram for describing a method for providing lung information using lung sound as a graph according to one embodiment of the inventive concept.

Referring to FIG. 13, the lung information acquisition method using the lung sound according to one embodiment of the inventive concept includes acquiring, in real-time, lung sound measurement data of a specific patient S610, acquiring processed lung sound measurement data using the lung sound measurement data S630, and creating and providing a variation graph of the processed lung sound measurement data S650.

The real-time acquisition of the lung sound measurement data of the specific patient S610 includes acquiring the lung sound measurement data using heart and lung sound data including the lung sound measurement data via the heart and lung sounds acquisition device inserted to a specific point of the patient's airway.

The heart and lung sounds acquisition device is configured to include a tube, a probe, a microphone and a connector. The tube is formed to extend from the heart and lung sounds measurement position in an esophagus or an air duct to an outside of the body. The probe is disposed at a first end of the tube and is placed in the heart and lung sounds measurement position to collect heart and lung sounds through the esophagus or air duct surface. The microphone converts the heart and lung sounds collected from the probe into electrical signals, and is placed inside the probe. The connector is connected to a cable at a second end of the tube, and transmits the electrical signals corresponding to the heart and lung sounds to a cardiovascular information output device.

In step S630 of acquiring the processed lung sound measurement data using the lung sound measurement data, at least one data included in the processed lung sound measurement data is acquired.

In one embodiment, in step S650 of creating and providing the variation graph of the processed lung sound measurement data, the variation graph of the processed lung sound measurement data refers to a real-time variation graph of the processed lung sound measurement data, and has the same variation tendency as that of a specific lung information data graph.

Conventionally, invasive measurement data may be obtained only when a tool is invasively inserted (e.g., a tool is inserted to the heart to determine a stroke volume). Thus, the variation in data could not be identified in surgery where it is difficult to invasively insert the tool.

According to the inventive concept, even without directly providing a lung information data graph, providing only the variation graph of the processed lung sound measurement data may allow providing the same variation tendency of the lung information data to a patient, a doctor, or a user who needs lung information.

In another embodiment, in step S650 of creating and providing the variation graph of the processed lung sound measurement data, the processed lung sound measurement data refers to frequency region data of the lung sound measurement data, and the variation graph of the processed lung sound measurement data includes at least one of a power variation based on a frequency band or a combination of frequency band differences.

According to the inventive concept, even when the lung information data value or graph is not directly provided, the lung information may be provided to the medical staff by providing, thereto, the variation graph of the processed lung sound measurement data, that is, at least one of the variation of the power based on the frequency band or the combination of frequency band differences.

For example, when determining presence or absence of secretion in the air duct with a stethoscope, auscultation should be repeated at intervals of about 10 to 30 minutes. Further, subtle variations of sounds should be detected using a hearing sense for diagnosis.

However, it is possible to determine the presence or absence of secretion in the air duct by reinforcing or attenuating a specific region that may be identified using continuous monitoring of heart and lung sounds and frequency band analysis of heart and lung sounds.

Therefore, according to the inventive concept, the medical staff may recognize the presence or absence of secretion in the air duct using the variation graph of the processed lung sound measurement data and appropriately cope with the same.

Further, the medical staff may recognize not only the presence or absence of secretion in the air duct, but also the lung water amount, mucus plug in the air duct, sputum in the air duct, the pulmonary alveolus opening and closing pressure and pulmonary edema, etc. based on the variation graph of the processed lung sound measurement data, and may appropriately cope with the same

FIG. 14 is a diagram for describing a method for calculating a calculated value of lung information using a lung sound and providing the calculated value according to one embodiment of the inventive concept.

Referring to FIG. 14, the method for calculating the calculated value of lung information and providing the calculated value according to one embodiment of the inventive concept include performing real-time acquisition of lung sound measurement data of a specific patient S710, acquiring processed lung sound measurement data using the lung sound measurement data S730, calculating a scale factor between the processed lung sound measurement data and lung information data value S750, and calculating the calculated value of the lung information data based on the processed lung sound measurement data and the scale factor, and providing the calculated value S770.

The step S710 of acquiring the lung sound measurement data of a specific patient in real time and step S730 of acquiring the processed lung sound measurement data using the lung sound measurement data are the same as described above in FIG. 13.

Acquiring the scale factor between the processed lung sound measurement data and the lung information data value S750 includes acquiring the scale factor for calculating the lung information data value using the processed lung sound measurement data. The step for acquiring the scale factor includes acquiring the scale factor using a graph or a matrix created based on the lung sound and lung invasive measurement data of a plurality of patients as previously acquired. A specific method for acquiring the scale factor using the graph or the matrix will be described later.

In one embodiment, calculating the calculated value of the lung information data based on the processed lung sound measurement data and the scale factor and providing the calculated value S770 includes creating a graph of lung information data based on the processed lung sound measurement data and the scale factor, and calculating and providing an exact value at each point on the graph as the calculated value of lung information data. In another embodiment, without creating the graph of lung information data, the exact value of lung information data may be calculated and provided.

FIG. 15 is a diagram for describing a method for deriving a correlation between reference lung sound data and lung invasive measurement data according to the inventive concept.

Referring to FIG. 15, the lung information acquisition method using the lung sound according to one embodiment of the inventive concept includes deriving a correlation between processed reference lung sound data and lung invasive measurement data S600, performing real-time acquisition of lung sound measurement data of a specific patient S610, acquiring processed lung sound measurement data using the lung sound measurement data S630, and creating and providing a variation graph of the processed lung sound measurement data S650.

Deriving the correlation between the processed reference lung sound data and lung invasive measurement data S600 occurs before the acquisition of the patient's lung sound measurement data measured in real time. The correlation is derived using the processed reference lung sound data and lung invasive measurement data that have been previously acquired from a plurality of other patients.

The reference lung sound data and the lung invasive measurement data may be obtained from at least one the same patient among the plurality of other patients and at the same time and may constitute a pair. The correlation between the processed reference lung sound data obtained by processing the reference lung sound data and the lung invasive measurement data is derived.

Further, in FIG. 15, it is shown that step S600 of deriving the correlation between the processed reference lung sound data and the lung invasive measurement data occurs before step S610 of acquiring the lung sound measurement data of a specific patient in real time to step S650 of creating and providing the variation graph of the processed lung sound measurement data. Further, step S600 of deriving the correlation between the reference lung sound data and the lung invasive measurement data may occur before step S710 of real-time acquisition of the specific patient's lung sound measurement data to step S770 of calculating the calculated value of the lung information data based on the processed lung sound measurement data and the scale factor, and providing the calculated value in FIG. 14.

Hereinafter, a method for acquiring lung information using the lung sound according to the inventive concept is described based on a graph showing the correlation as the embodiment of the correlation between the processed lung sound measurement data and the lung information data.

FIG. 16 is a graph to allow identification of presence or absence of secretion in an air duct based on a power variation based on a lung sound frequency band according to one embodiment of the inventive concept.

Referring to FIG. 16, in a graph of a secretion state in the air duct, a normal patient state (Clean) 29, a state before removing the secretion in the air duct of a disease patient (Pre-suction) 33 and a state after removing the secretion in the air duct of a disease patient (Post-suction) 31 are shown based on a frequency band.

On the graph of the frequency band, in the state before the secretion in the air duct of the disease patient is removed, an overall power is slightly stronger than that in the normal patient state. The state after removal of the secretion in the air duct of the disease patient has almost the same power as that in the normal patient state.

Therefore, comparing a current state of the secretion in the air duct with the graph of the normal patient using the processed lung sound measurement data, that is, the graph of the frequency region may allow immediately identifying the current state of the secretion in the air duct.

FIG. 17 is a diagram for describing a method for extracting lung information data using a correlation and lung sound measurement data according to the inventive concept.

Referring to FIG. 17, the lung information acquisition method using the lung sound of the inventive concept includes deriving a correlation between processed reference lung sound data and lung invasive measurement data S600, performing real-time acquisition of lung sound measurement data of a specific patient 5610, preforming acquisition of processed lung sound measurement data using the lung sound measurement data S630, creating and providing a real-time variation graph of the processed lung sound measurement data S650, and extracting lung information data using the correlation and the lung sound measurement data S670.

In one embodiment, in step S670 of extracting the lung information data using the correlation and the lung sound measurement data, the correlation is derived using a graph.

In another embodiment, extracting the lung information data may be performed using a learning algorithm based on the reference lung sound data and the lung invasive measurement data.

As described above in FIG. 15, in FIG. 17, it is shown that the step of extracting the lung information data using the correlation and the lung sound measurement data S670 occurs after step S600 of deriving the correlation between the processed reference lung sound data and the lung invasive measurement data to step S650 of creating and providing the variation graph of the processed lung sound measurement data. In another embodiment, deriving the correlation between the processed reference lung sound data and the lung invasive measurement data S600 may occur after preforming the real-time acquisition of lung sound measurement data of a specific patient S710 to calculating the calculated value of lung information data based on the processed lung sound measurement data and the scale factor, and providing the calculated value S770.

FIGS. 18 and 19 are diagrams for describing a method for removing noise in deriving a correlation between reference lung sound data and lung invasive measurement data according to the inventive concept.

The step of deriving the correlation as described above in FIG. 15 may further include a step of removing noise.

Referring to FIG. 18, the step of removing the noise may include acquiring reference lung data S810, acquiring a feature of reference lung sound data using a feature filter S830, matching the reference lung data with the reference lung sound data S850, and upon determination that an abnormal region is included in a wave of the reference lung data, excluding reference lung sound data matching the abnormal region as noise S870.

The reference lung data may refer to data for reference in removing the noise when deriving the correlation between the reference lung sound data and the lung invasive measurement data, among data acquired in the non-invasive measurement manner.

The reference lung data may be used to identify the abnormal region in real time. The abnormal region refers to a region in which noise is created in the heart and lung sound data due to the patient's movement and posture change during surgery. Because it is difficult to determine the abnormal region using only the reference lung sound data, the abnormal region may be identified using the reference lung data, and a portion of the reference lung sound data matching the abnormal region may be excluded, thereby to achieve an effect of accurately deriving the correlation between the processed reference lung sound data and the lung invasive measurement data.

Further, referring to FIG. 19, the step of removing the noise includes step S910 of converting a graph on a time region of the correlation into a graph on a frequency region, and upon determination that the abnormal region is included in the graph on the frequency region, step S930 of excluding a portion of the time region matching the abnormal region as noise.

In determining whether the abnormal region is included in the graph on the frequency region, when a value of a specific region exceeds a range of a predetermined value in the graph on the frequency region, the specific region is determined as the abnormal region.

Because it is difficult to determine the abnormal region in the graph on the time region, the abnormal region may be identified in the graph on the frequency region, and a portion of the time region matching the abnormal region may be excluded, thereby to achieve an effect of accurately deriving the correlation between the processed reference lung sound data and the lung invasive measurement data.

FIG. 20 is a diagram for describing a method for creating a matrix for extracting a scale factor according to the inventive concept.

A front step of the method for calculating the calculated value of the lung information using the lung sound and providing the calculated value, as described above in FIG. 14 may further include a step of creating a matrix for calculating a scale factor using reference lung sound data, and lung invasive measurement data as previously acquired from a plurality of patients.

Referring to FIG. 20, the step for creating the matrix for extracting the scale factor includes acquiring reference lung sound data, and acquiring processed reference lung sound data using the reference lung sound data S1010, acquiring lung invasive measurement data S1030, calculating a scale factor between the processed reference lung sound data and the lung invasive measurement data S1050, and creating a matrix using the processed reference lung sound data, the lung invasive measurement data and the scale factor S1070.

In step S1010 of acquiring the reference lung sound data and acquiring the processed reference lung sound data using the reference lung sound data, the reference lung sound data refers to lung sound data previously acquired from a plurality of patients, and the processed reference lung sound data refers to processed data as calculated from the reference lung sound data or some values derived therefrom.

The reference lung sound data and the lung invasive measurement data may be obtained from at least one the same patient among the plurality of other patients and at the same time and may constitute a pair. The scale factor between the processed reference lung sound data obtained by processing the reference lung sound data and the lung invasive measurement data is derived.

In step S1050 of calculating the scale factor between the processed reference lung sound data and the lung invasive measurement data, the scale factor between the reference lung sound data and the lung invasive measurement data as obtained from at least one the same patient among the plurality of other patients and at the same time may be calculated and acquired.

The step S1070 of creating the matrix using the processed reference lung sound data, the lung invasive measurement data and the scale factor may include creating the matrix using the processed reference lung sound data, the lung invasive measurement data, and the scale factor for a physical condition of each of a plurality of patients.

Creating the matrix using the processed reference lung sound data, the lung invasive measurement data and the scale factor for the physical condition of each of the plurality of patients may allow a scale factor that matches the patient's body condition and lung sound data on the matrix to be acquired in subsequently calculating the lung information data value using only the patient's lung sound measurement data. Further, in this case, not only the scale factor may be acquired, but lung invasive measurement data may also be acquired.

Therefore, a front step of the method for calculating the calculated value of lung information using the lung sound and providing the calculated value, as described above in FIG. 14 may further include the step of creating the matrix for calculating the scale factor using reference lung sound data, and lung invasive measurement data as previously acquired from a plurality of patients. Acquiring the scale factor between the processed lung sound measurement data and the lung information data value S750 may include acquiring the scale factor using the created matrix.

Thereafter, in step S770 of calculating the calculated value of lung information data based on the processed lung sound measurement data and the scale factor and providing the calculated value, the reference lung sound data on the matrix may be compared with initial lung sound measurement data acquired from the patient and the calculated value may be accurately calculated based on the comparison result.

In one embodiment, when acquiring a certain portion of the lung sound measurement data, and acquiring only one accurate calculated value of the lung information data matching the body condition of the patient matching a portion of the reference lung sound data matching the certain portion, all of the calculated values of lung information data may be continuously acquired.

Further, calculating the scale factor between the processed reference lung sound data and the lung invasive measurement data S1050 may be performed using a learning algorithm. The step S1070 of creating the matrix using the processed reference lung sound data, the lung invasive measurement data, and the scale factor may be performed using a learning algorithm, thereby providing the matrix using the scale factor to a user.

A lung information acquisition device using a lung sound according to another embodiment of the inventive concept includes a lung sound measurement data acquisition unit that acquires real-time lung sound measurement data of a specific patient, a processed lung sound measurement data acquisition unit for acquiring at least one processed lung sound measurement data using the lung sound measurement data, and a processed lung sound measurement data variation graph providing unit to create and provide a variation graph of the processed lung sound measurement data. The variation graph of the processed lung sound measurement data represents specific lung information data.

The lung information acquisition device according to the inventive concept has the same configuration as that of the lung information acquisition method described in FIGS. 13 to 20.

Further, when describing an overall configuration of the inventive concept in more detail, the lung information acquisition method using the lung sound according to one embodiment of the inventive concept includes a step in which a computer acquires lung sound measurement data of a specific patient in real time, a step in which the computer acquires at least one processed lung sound measurement data using the lung sound measurement data, and a step in which the computer creates a variation graph of the processed lung sound measurement data and provides the lung information data.

The lung information acquisition method using lung sound according to one embodiment of the inventive concept includes a step in which a computer acquires lung sound measurement data of a specific patient in real time, a step in which the computer acquires at least one processed lung sound measurement data using the processed lung sound measurement data, a step in which the computer calculates a scale factor between the processed lung sound measurement data and lung information data value, and a step in which the computer calculates a calculated value of specific lung information data based on the processed lung sound measurement data and the scale factor, and provides the calculated value.

The processed lung sound measurement data refers to frequency region data of the lung sound measurement data. The lung information data includes one or more selected from a group consisting of the lung water amount, secretion in the air duct, mucus plug in the air duct, sputum in the air duct, a pulmonary alveolus opening and closing pressure and lung edema of the patient.

The variation graph of the processed lung sound measurement data includes at least one of a variation of a power based on a frequency band or a combination of frequency band differences. The variation graph of the processed lung sound measurement data has the same tendency of variation as that of the specific lung information data graph.

The step for acquiring, by the computer, the scale factor between the processed lung sound measurement data and the lung information data value includes acquiring the scale factor using a graph or a matrix created based on the lung sound and lung invasive measurement data of a plurality of patients as previously acquired.

Calculating, by the computer, the calculated value of the specific lung information data based on the processed lung sound measurement data and the scale factor and providing the calculated value includes creating, by the computer, a graph of lung information data based on the processed lung sound measurement data and the scale factor, and calculating and providing, by the computer, an exact value at each point on the graph as the calculated value of lung information data. In another embodiment, without creating the graph of lung information data, the exact value of lung information data may be calculated and provided by the computer.

A lung information acquisition method using a lung sound according to one embodiment of the inventive concept may further include, before the step of acquiring, by the computer, the lung sound measurement data of the specific patient in real time, a step of deriving, by the computer, a correlation between the processed reference lung sound data and the lung invasive measurement data.

A lung information acquisition method using a lung sound according to one embodiment of the inventive concept may further include, after the step of creating, by the computer, the variation graph of the processed lung sound measurement data, and providing, by the computer, the lung information data, a step in which the computer extracts lung information data using the correlation between the processed reference lung sound data and the lung invasive measurement data and the lung sound measurement data.

A lung information acquisition method using a lung sound according to one embodiment of the inventive concept may further include, after the step of calculating, by the computer, the calculated value of specific lung information data based on the processed lung sound measurement data and the scale factor and providing, by the computer, the calculated value, a step of extracting, by the computer, lung information data using the correlation between the processed reference lung sound data and lung invasive measurement data and the lung sound measurement data.

The correlation may be derived using a graph or may be extracted using a learning algorithm based on the processed reference lung sound data and lung invasive measurement data.

The step of deriving the correlation may further include a step of removing noise. The step of removing the noise may include acquiring, by the computer, reference lung data, acquiring, by the computer, a feature of the reference lung sound data using a feature filter, matching, by the computer, the reference lung data with the reference lung sound data from which the feature is acquired, and upon determination that an abnormal region is included in a wave of the reference lung data, excluding, by the computer, reference lung sound data matching the abnormal region as noise.

The step in which the computer calculates a calculated value of specific lung information data based on the processed lung sound measurement data and the scale factor, and provides the calculated value includes a step of, by the computer, creating a matrix for calculating the scale factor using reference lung sound data and lung invasive measurement data acquired previously from a plurality of patients. The step of creating, by the computer, the matrix for extracting the scale factor includes a step of acquiring, by the computer, reference lung sound data and acquiring, by the computer, at least one processed reference lung sound data using the reference lung sound data, a step of acquiring, by the computer, lung invasive measurement data, a step of calculating, by the computer, a scale factor between the processed reference lung sound data and the lung invasive measurement data, and a step of creating, by the computer, a matrix using the processed reference lung sound data, the lung invasive measurement data and the scale factor.

The reference lung sound data refers to lung sound data previously acquired from a plurality of patients, and the processed reference lung sound data refers to processed data as calculated from the reference lung sound data or some values derived therefrom.

In the step of calculating, by the computer, the scale factor between the processed reference lung sound data and the lung invasive measurement data, the scale factor between the reference lung sound data and the lung invasive measurement data as obtained from at least one the same patient among the plurality of other patients and at the same time may be calculated and acquired by the computer.

The step of creating, by the computer, the matrix using the processed reference lung sound data, the lung invasive measurement data and the scale factor may include creating, by the computer, the matrix using the processed reference lung sound data, the lung invasive measurement data, and the scale factor for a physical condition of each of a plurality of patients.

A lung information acquisition program using a lung sound according to another embodiment of the inventive concept is stored in a computer-readable recording medium to execute any one of the above methods.

A lung information acquisition device using a lung sound according to another embodiment of the inventive concept includes a lung sound measurement data acquisition unit that acquires lung sound measurement data of a specific patient in real time, a lung sound processed data acquisition unit that acquires at least one processed lung sound measurement data using the lung sound measurement data, and a processed lung sound measurement data variation graph providing unit that creates and provides a variation graph of the processed lung sound measurement data.

A lung information acquisition device using a lung sound according to another embodiment of the inventive concept includes a lung sound measurement data acquisition unit that acquires lung sound measurement data of a specific patient in real time, a lung sound processed data acquisition unit that acquires at least one processed lung sound measurement data using the lung sound measurement data, a scale factor calculating unit for calculating a scale factor between the processed lung sound measurement data and lung information data value, and a lung information data value calculating unit that calculates a calculated value of the specific lung information data based on the processed lung sound measurement data and the scale factor and provides the calculated value.

In FIGS. 21 to 27, a description of a method for acquiring heart and lung sound signals will be made. The method for acquiring the heart and lung sound signals may be applied to the method for acquiring using the heart and lung sounds acquisition device.

FIG. 21 is a flow chart showing a method for acquiring heart and lung sound signals according to one embodiment of the inventive concept. The method in FIG. 21 is performed by a computer.

Referring to FIG. 21, the computer may acquire a heart sound and/or a lung sound based on a measurement position of a probe S1100.

In this connection, the probe refers to a device for measuring the heart sound and/or the lung sound from a patient, and may be a stethoscope. For example, the probe (e.g., the stethoscope) may be inserted into the patient's esophagus to acquire the heart sound and/or the lung sound at the inserted position. In another example, it is possible to measure the patient's heart sound and/or lung sound without the probe being inserted into the esophagus. The patient's heart sound and/or lung sound may be acquired through the esophagus as well as other body parts. Further, the heart sound and/or the lung sound may be acquired from the probe and then other types of acoustic signal processing may be performed.

In one embodiment, the computer may determine at least one measurement position of the probe for measuring the patient's heart sound and/or lung sound and may acquire the heart sound and/or the lung sound at each of the at least one determined measurement position of the probe. For example, while the probe is inserted into the patient's esophagus, a plurality of measurement positions for measuring the heart sound and/or the lung sound may be determined based on an insertion path. Alternatively, a plurality of measurement positions may be determined based on a path along which the probe is gently removed after the probe is completely inserted into the patient's esophagus.

In acquiring the heart sound and/or the lung sound at each measurement position of the probe, the heart sound and the lung sound may be acquired from the probe at once. In this connection, when percentages of the heart sound and the lung sound to be acquired are different from each other, the percentages of the heart sound and the lung sound as acquired may be adjusted using a microphone. For example, when a position of the microphone and the number of microphones are adjusted during measurement of the heart sound and the lung sound, a difference between signal types of the heart sound and the lung sound may occur. Thus, the percentages of the heart sound and the lung sound as acquired may be controlled to be different from each other, based on the difference. In this connection, the microphone may be connected to the probe or included in the probe itself. Alternatively, the microphone may be a separate component from the probe, and, if necessary, it may be used together with the probe for heart sound and/or lung sound acquisition.

According to an embodiment, the heart sound and lung sound acquired from the probe at one time may be subjected to filtering using a difference in a frequency band and may be separated from each other. Further, the heart sound may be separated into a first heart sound (S1) and a second heart sound (S2) based on a amplitude or a shape of the waveform of the signal thereof. The lung sound may be separated into left and right lung sounds based on a frequency band of the signal thereof.

The computer may analyze the heart sound and/or the lung sound acquired based on the measurement position of the probe and determine a target measurement position of the probe based on the analysis result S1110.

In one embodiment, the computer may extract a signal waveform of the heart sound and/or the lung sound acquired at each of at least one measurement position, and analyze the signal waveform of each of the extracted heart sound and/or lung sound, and determine whether the corresponding signal waveform of the heart sound and/or lung sound is an appropriate signal waveform. In this connection, the computer may determine whether the heart sound and/or the lung sound acquired at the corresponding measurement position is a suitable signal, based on the amplitude variation and the amplitude of the signal waveform of the heart sound and/or the lung sound. When the signal waveform of the heart sound and/or lung sound is determined to be the appropriate signal waveform based on the determination result, the computer may derive the measurement position of the probe corresponding to the appropriate signal waveform, and may determine the target measurement position of the probe based on the derived measurement position. For example, the computer may analyze the signal waveform of the heart sound and/or the lung sound at each measurement position acquired from the probe and determine the heart sound and/or lung sound having the most suitable signal waveform, and may determine the measurement position of the probe corresponding to the most suitable signal waveform as the target measurement position.

The computer may provide information on the target measurement position of the probe S1120. In one embodiment, the computer may output the target measurement position of the probe on a screen, or may provide the information to a measurer (e.g., a medical staff) who measures the heart sound and/or the lung sound via various methods of guiding the probe to the target measurement position. For example, the computer may compare the target measurement position with a current measurement position of the probe and guide the probe to the target measurement position.

The computer may finally acquire the patient's heart sound and/or lung sound from the probe at the target measurement position, and may pre-process the heart sound and/or lung sound which in turn may be used in various manners. That is, the computer may collect the heart sound and/or the lung sound of the patient acquired at the target measurement position, and may construct a data set. This data set of the heart sound and/or the lung sound may be used to construct a learning model. When the learning model is constructed in this way, the computer may acquire the heart sound and/or the lung sound measured at the target measurement position from a new patient and may apply the same to the learning model and identify and guide a new health status of the patient in real time. In an embodiment, a feature based analysis or a deep learning based analysis may be performed based on the heart sound and/or the lung sound acquired at the target measurement position. This will be described in detail later with reference to FIGS. 24 to 26.

Further, the computer may finally acquire the patient's heart sound and/or lung sound from the probe at the target measurement position, and then may apply a method of FIG. 23 to be described later to the finally acquired heart sound and/or lung sound.

FIGS. 22A to 22D are diagrams showing a signal waveform of the heart sound and/or the lung sound acquired based on the measurement position of the probe. Each graph shown in FIGS. 22A to 22D represents each of an ECG (top of each graph) and heart and lung sound signals (bottom of each graph) as measured based on the position of the probe inserted into the patient's esophagus.

According to FIG. 22A, a first signal waveform as measured when the probe is inserted into the esophagus and is placed at a position (first measurement position) of a depth of 38 cm spaced from a incisor is shown. In this case, the computer may determine whether the first signal waveform corresponds to a suitable signal, based on the amplitude variation or the amplitude of the first signal waveform. As shown in FIG. 22A, it may be seen that the first heart sound is dominant in the first signal waveform, while the second heart sound is very faint and weak in intensity in the first signal waveform. Accordingly, the computer may determine that the first signal waveform is an inappropriate signal waveform.

According to FIG. 22B, a second signal waveform as measured when the probe is inserted into the esophagus and is placed at a position (second measurement position) of a depth of 32 cm spaced from the incisor is shown. In this case, the computer may determine whether the second signal waveform corresponds to a suitable signal based on the amplitude variation or the amplitude of the second signal waveform. As shown in FIG. 22B, both the first heart sound and the second heart sound are derived from the second signal waveform. However, in the second signal waveform, the first heart sound is dominant and has a greater amplitude, compared to the second heart sound. Accordingly, the computer may determine that the second signal waveform is an inappropriate signal waveform.

According to FIG. 22C, a third signal waveform as measured when the probe is inserted into the esophagus and is placed at a position (third measurement position) of a depth of 28 cm spaced from the incisor is shown. In this case, the computer may determine whether the third signal waveform corresponds to a suitable signal based on the amplitude variation or the amplitude of the third signal waveform. As shown in FIG. 22C, both the first heart sound and the second heart sound are clearly derived from the third signal waveform. It may be seen that the first heart sound and the second heart sound have a relatively similar pattern in terms of the amplitude or variation of the amplitude. Therefore, the computer may determine that the third signal waveform is a suitable signal waveform.

According to FIG. 22D, a fourth signal waveform as measured when the probe is inserted into the esophagus and is placed at a position (fourth measurement position) of a depth of 25 cm spaced from the incisor is shown. In this case, the computer may determine whether the fourth signal waveform corresponds to a suitable signal based on the amplitude variation or the amplitude of the fourth signal waveform. As shown in FIG. 22D, both the first heart sound and the second heart sound are derived from the fourth signal waveform, and the first heart sound and the second heart sound have relatively similar pattern in terms of the amplitude or variation of the amplitude. However, it may be seen that the amplitude of the fourth signal waveform is reduced compared to that of the third signal waveform. Accordingly, the computer may determine that the fourth signal waveform is an unsuitable signal waveform compared to the third signal waveform.

That is, the computer may acquire the signal waveforms (first to fourth signal waveforms) of the heart sound and/or the lung sound, respectively, at the measurement positions of the probe (first to fourth measurement positions), and may analyze the signal waveforms (first to fourth signal waveforms) of the acquired heart sound and/or lung sound, respectively, and may derive the most suitable signal waveform (e.g., the third signal waveform). Further, the computer may determine the measurement position (third measurement position) of the probe corresponding to the derived most suitable signal waveform (e.g., the third signal waveform) as the target measurement position. In other words, the computer determines the position 28 cm deep from the incisor as the target measurement position, and may provide the position information corresponding thereto.

FIG. 23 is a flow chart showing a method for acquiring heart and lung sound signals according to another embodiment of the inventive concept. The method of FIG. 23 is performed by a computer.

Referring to FIG. 23, the computer may acquire a heart sound and/or a lung sound from the probe S1200.

According to an embodiment, the computer may acquire the heart sound and/or the lung sound according to the method for FIG. 21. That is, the computer may determine the target measurement position of the probe based on the heart sound and/or lung sound acquired at the measurement position of the probe, and then may finally acquire and use the heart sound and/or the lung sound measured from the probe at the determined target measurement position.

The computer may analyze the acquired heart sound and/or lung sound and determine whether an abnormal region is included therein S1210.

In one embodiment, the computer may extract and pre-store the heart sound and/or the lung sound including the abnormal region from a plurality of patients, or may learn the heart sound and/or the lung sound based on deep learning to construct training data. Thereafter, the computer may determine whether or not the abnormal region exists in the heart sound and/or the lung sound acquired in step S1200, based on the training data as previously stored or constructed to allow the abnormal region pattern to be identified. In another example, a person (e.g., a medical staff) may directly determine whether there is the abnormal region in the heart sound and/or the lung sound.

Upon determination that the abnormal region is included in the heart sound and/or the lung sound, based on the determination result, the computer may correct the signal waveform of the abnormal region based on a predefined normal signal waveform S1220.

In one embodiment, the computer may remove a portion corresponding to the abnormal region from the signal waveform of the heart sound and/or lung sound, and may replace a predefined normal signal waveform into the removed portion. The predefined normal signal waveform may include a portion of a signal waveform of the heart sound and/or the lung sound acquired from the patient as determined as a normal region. Alternatively, the predefined normal signal waveform may include a normal signal waveform of a heart sound and/or a lung sound pre-acquired from other patients. Alternatively, a predetermined reference signal waveform of a heart sound and/or a lung sound may be used as the predefined normal signal waveform. For example, when the computer analyzes the heart sound and/or the lung sound acquired from the patient and thus determines that there is a signal waveform portion caused by a valve abnormality, this portion may be recognized as an abnormal region and correction may be performed to remove the signal waveform portion as caused by the valve abnormality or the like. Further, the computer may insert a normal signal of a heart sound and/or a lung sound into the removed portion, and may provide data that may allow accurately grasping a variation before and after the surgery or a variation occurring during the surgery.

In another embodiment, upon determination that the abnormal region is included in the lung sound based on the signal waveform of the heart sound and/or the lung sound, the computer may perform correction to remove all signals corresponding to the lung sound and acquire only signals corresponding to the heart sound. For example, a patient with bronchiectasis may have a problem with the lung sound, but has no problem with the heart sound. In this case, a normal heart sound signal may be acquired by performing correction to remove only the lung sound. In this connection, for a patient with bronchiectasis, a high frequency band is generated according to a breathing cycle. Thus, the computer may acquire a normal signal by performing correction to remove the high frequency band through filtering. Otherwise, upon determination that the abnormal region is included in the heart sound based on the signal waveform of the heart sound and/or the lung sound, the computer may perform correction to remove all signals corresponding to the heart sound and acquire only the signals corresponding to the lung sound. For example, when in the signal waveform of the heart sound and/or lung sound, a continuous heart noise occurs after the first heart sound, the computer may acquire only a lung sound signal by performing correction to remove an entirety of the heart sound, or may perform correction to remove only the heart sound noise region for use in analyzing the first heart sound.

According to steps S1200 to S1220, it is possible to acquire a heart sound signal and/or a lung sound signal in a normal state from which the abnormal region has been removed.

The computer may monitor a health status of the patient based on the heart sound and/or the lung sound (a signal waveform from which an abnormal region has been removed) including the corrected signal waveform acquired in step S1220.

Further, the computer may track the variation of the signal waveform of the abnormal region in the heart sound and/or lung sound including the abnormal region, and may predict the patient's disease in real time based on the tracked variation of the signal waveform for the abnormal region.

Further, the computer may perform a pre-processing process based on the heart sound and/or the lung sound including the corrected signal waveform acquired in step S1220, and use the pre-processed data in various manners. That is, the computer may acquire the heart sound and/or the lung sound in the normal state from which the abnormal region has been removed via correction, and then may construct a data set based on the same. The data set may be subsequently used to create a learning model via learning. For example, a feature based analysis or deep learning based analysis may be performed. This will be described in detail later with reference to FIGS. 24 to 26.

According to an embodiment, the computer may measure a signal quality of a heart sound and/or a lung sound. In this connection, when the measured signal quality is compared with a predefined threshold and then the heart sound and/or the lung sound is determined as an abnormal signal based on the comparison result, the computer does not perform analysis on the heart sound and/or the lung sound, such as a process of determining whether the abnormal region is included in the heart sound and/or the lung sound, after a time when the abnormal signal occurs. For example, the computer may measure the signal of the heart sound and/or the lung sound using a device measuring the signal quality (e.g., BIS), and may measure a signal quality index (SQI) as the signal quality of the measured signal. When the SQI value drops below a predefined threshold (e.g., when a patient movement occurs during heart sound and/or lung sound measurement), the computer may not proceed with the analysis on the heart sound and/or the lung sound because it determines that a problem has occurred in the quality of the heart sound and/or the lung sound. Therefore, in accordance with the inventive concept, when the heart sound and/or the lung sound contains the abnormal region or there is a quality problem thereof, only the normal signal excluding the abnormal signal may be acquired, thereby to obtain a more accurate analysis result of the patient's health state or the heart sound and/or the lung sound.

In one example, as described above, the heart sound and/or the lung sound acquired at the target measurement position (first acquired heart sound and/or lung sound), or the heart sound and/or the lung sound (second acquired heart sound and/or lung sound) acquired via the pre-processing correction may be used as training data for the feature based analysis or deep learning based analysis. Further, the computer may construct a training data set by performing supervised learning or non-supervised learning of the training data. Then, the computer may perform analysis and prediction on newly added first and second acquired heart sound and/or lung sound, based on the training data set.

For example, the feature based analysis may include a supervised learning method. When the feature based analysis is applied, features of the first and second acquired heart sound and/or lung sound may be extracted from the training data set learned by the user.

The deep learning based analysis may include a non-supervised learning method. In the deep learning based analysis, a training data set may be established using a non-supervised learning method by matching the first and second acquired heart sounds and/or lung sounds of a plurality of patients with invasively acquired reference data (e.g. gold standard data). Then, the computer may apply the established deep learning-based training data set to learn the correlation between specific reference data (e.g., gold standard data) and specific factors in the first and second acquired heart sounds and/or lung sounds.

Further, as described above, when the learning model is constructed via a learning method such as supervised learning or non-supervised learning of the first and second acquired heart sound and/or lung sound of patients, the computer may predict the variation in health status of a new patient based on this learning model.

For example, when performing surgery on a new patient, the computer may acquire the first and second acquired heart sounds and/or lung sounds from the new patient, and may apply the first and second acquired heart sounds and/or lung sounds to the learning model, such that the patient's state variation may be identified. In this case, the variation of the patient's condition may be grasped only when data having the same condition as that of the heart sound and/or lung sound used when constructing the learning model is acquired.

Further, when the patient's first and second acquired heart sounds and/or lung sounds are acquired and applied to the learning model, a data value to be acquired invasively or non-invasively may be inferred therefrom. Thus, the variation of the patient's condition during or after surgery may be identified.

Further, the computer may set the target measurement position before the patient's surgery. Then, the heart sounds and/or lung sounds may be acquired at the set target measurement position. Therefore, the computer may determine whether there is a signal waveform that needs correction in the heart sound and/or lung sound acquired before the surgery, and may perform correction thereon, and may separate the heart sound and the lung sound from each other. This separation may be done based on a difference between the heart sound period and the lung sound period. Thereafter, variations in factors (e.g., STI, S2 amplitude, etc.) acquired from the heart sound data and the lung sound data as separated from each other may be applied to the learning model to determine the patient's condition in real time.

FIGS. 24 to 26 are diagrams for describing an example of utilizing the acquired heart sound and/or lung sound according to an embodiment of the inventive concept.

The heart sound and/or the lung sound acquired according to the embodiment of the inventive concept may be used as pre-processing target data during feature based analysis or deep learning based analysis. In an embodiment, in the feature based analysis, the heart sound and/or the lung sound acquired according to the embodiment of the inventive concept may be used as an index for predicting a bio signal that may be acquired invasively during or before and after surgery. In the deep learning based analysis, feature elements may be extracted from the heart sound and/or the lung sound acquired according to the embodiment of the inventive concept. Then, the computer may create training data and a training model learned based on deep learning while using the extracted feature elements as inputs. Hereinafter, examples in which the heart sound and/or the lung sound acquired according to the inventive concept is used as an index for predicting other data will be described.

FIG. 24 is a graph showing a ventricular systolic time 35 and the pulse pressure 19 included in the heart sound and/or the lung sound acquired according to an embodiment of the inventive concept. As shown in FIG. 24, it may be seen that there is a correlation between the variation of the ventricular systolic 35 and the pulse pressure 19. Therefore, the computer may calculate the pulse pressure 19 using the ventricular systolic time 35 acquired according to the embodiment of the inventive concept. Further, the computer may derive a systolic time interval (STI) from the heart sound acquired according to the embodiment of the inventive concept, and may determine the pulse pressure variation (PPV) based on the STI.

FIG. 25 is a graph showing a ventricular systolic time 37 and the stroke volume 17 included in the heart sound and/or the lung sound acquired according to an embodiment of the inventive concept. As shown in FIG. 25, it may be seen that there is a correlation between variation tendencies of the ventricular systolic time 37 and the stroke volume 17. Therefore, the computer may calculate the ventricular systolic time interval 37 from the heart sound acquired according to the embodiment of the inventive concept, and then may post-correct the ventricular systolic time interval pattern to calculate the stroke volume.

FIG. 26 is a graph showing a second heart sound 39 and the systemic vascular resistance (SVR) 13 included in the heart sound and/or the lung sound acquired according to the embodiment of the inventive concept. As shown in FIG. 26, it may be seen that there is a correlation between variation tendencies of the second heart sound 39 and the systemic vascular resistance 13. Therefore, the computer may extract the second heart sound 39 from the heart sound acquired according to the embodiment of the inventive concept and then may post-process an amplitude of the second heart sound 39 to predict the systemic vascular resistance 13 in a non-invasive manner.

In FIGS. 24 to 26, the embodiments in which other data (e.g., a pulse pressure, a pulse pressure variation amount, a stroke volume, a systemic vascular resistance) having a correlation with the ventricular systolic time, the systolic time interval (STI), and the second heart sound S2 among various feature elements included in the heart sound and/or the lung sound are predicted based on the ventricular systolic time, the systolic time interval (STI), and the second heart sound S2 have been described. Those are just described by way of example. The inventive concept is not limited thereto.

That is, according to the inventive concept, when the heart sound and/or the lung sound acquired from the patient is applied to the learning model, various feature elements included in the heart sound and/or the lung sound may be extracted, and various data having correlations with the extracted feature elements may be derived. In this connection, conventionally, the derived data should be obtained only in an invasive measurement manner. However, in an embodiment of the inventive concept, even when only the heart sound and/or the lung sound from the patient is measured, the invasive data may be derived via learning in a non-invasive manner.

FIG. 27 is a block diagram showing a device that performs a method for acquiring heart and lung sound signals according to an embodiment of the inventive concept.

Referring to FIG. 27, a device 1300 may include an input unit 1310, a processor 1320, an output unit 1330, and a memory 1340.

The input unit 1310 may receive a heart sound and/or a lung sound from a probe (e.g., a stethoscope). The output unit 1330 may output various information, for example, information on a target measurement position of a probe. In one embodiment, the output unit 1330 may be executed by the processor 1320 to output the patient's body information determined from the heart sound and/or the lung sound acquired according to the embodiment of the inventive concept.

The processor 1320 performs a method for acquiring the heart and lung sound signals according to an embodiment of the inventive concept.

The memory 1340 may store a computer program by which a method for acquiring heart and lung sound signals according to an embodiment of the inventive concept is implemented.

The steps of the method or the algorithm described in relation to the embodiments of the inventive concept may be directly implemented in hardware, implemented using a software module executed by the hardware, or implemented by a combination thereof. The software module may reside on Random Access Memory (RAM), Read Only Memory (ROM), Erasable Programmable ROM (EPROM), Electrically Erasable Programmable ROM (EEPROM), Flash Memory, hard disk, removable disk, CD-ROM, or a computer-readable recording medium of any form well known in the technical field to which the inventive concept belongs.

According to the inventive concept, the patient's heart sound may be acquired in real time, and the tendency of the graph of the cardiovascular information and an accurate value thereof may be acquired using the heart sound.

Further, according to the inventive concept, acquiring the cardiovascular information using the patient's heart sound may allow the cardiovascular information to be acquired more rapidly than when acquiring the cardiovascular information in an invasive manner.

Further, according to the inventive concept, acquiring the reference heart sound data and invasive measurement data of various patients may allow creating the matrix for the scale factor necessary for deriving the accurate value of the cardiovascular information.

Further, according to the inventive concept, it is possible to acquire the patient's lung sound in real time and use the same to acquire the graph tendency and an accurate value of the lung information.

Further, according to the inventive concept, acquiring the lung information using the lung sound of the patient may allow the lung information to be acquired faster than when acquiring the lung information invasively.

Further, according to the inventive concept, acquiring the reference lung sound data and invasive measurement data of various patients may allow creating the matrix for the scale factor necessary for deriving an accurate value of the lung information.

According to the inventive concept, acquiring the heart and lung sounds with improved signal quality for each patient may allow accurately determining the patient's health status and disease diagnosis more effectively. Further, the heart and lung sounds having improved quality may be used as pre-processing target data, and thus may be used as an analysis index to predict various biological signals.

The effects of the inventive concept are not limited to the effects mentioned above. Other effects not mentioned will be clearly understood by those skilled in the art from the above descriptions.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. An information acquisition method using heart and lung sounds, the method comprising:
acquiring, by a computer, heart sound measurement data of a specific patient in real time;
acquiring, by the computer, at least one processed heart sound measurement data using the heart sound measurement data in real time;
extracting, by the computer, a scale factor between the processed heart sound measurement data and a cardiovascular data value; and
calculating and providing, by the computer, a calculated value of specific cardiovascular information data based on the processed heart sound measurement data and the scale factor, wherein the cardiovascular information data has the same variation tendency as a variation tendency of specific processed heart sound measurement data.

2. The method of claim 1, wherein the method further comprises, creating and providing, by the computer, a real-time variation graph of the processed heart sound measurement data,
wherein the real-time variation graph of the processed heart sound measurement data has the same variation tendency as a variation tendency of a specific cardiovascular information data graph.

3. The method of claim 1, wherein the processed heart sound measurement data includes a second heart sound maximum amplitude or a power,
wherein the cardiovascular information data includes a systemic vascular resistance (SVR).

4. The method of claim 1, wherein the processed heart sound measurement data includes a systolic time interval (S1-S2 interval, STI),
wherein the cardiovascular information data includes a stroke volume (SV), a pulse pressure (PP) or a pulse pressure variation (PPV).

5. The method of claim 1, wherein the processed heart sound measurement data includes a first heart sound maximum amplitude or a power,
wherein the cardiovascular information data includes a cardiac muscle contractility variation,
wherein the cardiac muscle contractility variation is calculated as a peak of a waveform obtained by differentiating an arterial pressure waveform in one heart period.

6. The method of claim 1, wherein the acquiring of the heart sound measurement data includes acquiring the heart sound measurement data using heart and lung sound data containing the heart sound measurement data via a heart and lung sounds acquisition device inserted to a specific point in an airway or an esophagus of the patient.

7. The method of claim 1, wherein the method further comprises deriving, by the computer, a correlation between processed reference heart sound data and cardiovascular invasive measurement data, using the processed reference heart sound data and the cardiovascular invasive measurement data,
wherein the processed reference heart sound data and the cardiovascular invasive measurement data are acquired from at least one the same patient and at the same time.

8. An information acquisition method using heart and lung sounds, the method comprising:
acquiring, by a computer, lung sound measurement data of a specific patient in real time;
acquiring, by the computer, at least one processed lung sound measurement data using the lung sound measurement data;
calculating, by the computer, a scale factor between the processed lung sound measurement data and a lung information data value; and
calculating and providing, by the computer, a calculated value of specific lung information data based on the processed lung sound measurement data and the scale factor.

9. The method of claim 8, wherein the method further comprises creating, by the computer, a variation graph of the processed lung sound measurement data to provide lung information data.

10. The method of claim 8, wherein the processed lung sound measurement data includes frequency region data of the lung sound measurement data,
wherein the lung information data includes at least one selected from a group consisting of a lung water amount, secretion in an air duct, mucus plug in the air duct, sputum in the air duct, a pulmonary alveolus opening and closing pressure and pulmonary edema.

11. The method of claim 9, wherein the variation graph of the processed lung sound measurement data includes at least one of a power variation based on a frequency band or a combination of frequency band differences,
wherein the variation graph of the processed lung sound measurement data has the same variation tendency as a variation tendency of a specific lung information data graph.

12. The method of claim 8, wherein calculating, by the computer, the scale factor between the processed lung sound measurement data and the lung information data value by the computer includes acquiring, by the computer, the scale factor using a graph or a matrix created based on a lung sound and lung invasive data previously acquired from a plurality of patients.

13. The method of claim 8, wherein the method further comprises, before acquiring, by the computer, the lung sound measurement data of the specific patient in real time, deriving, by the computer, a correlation between the processed reference lung sound data and lung invasive measurement data.

14. An information acquisition method using heart and lung sounds, the method comprising:
acquiring, by a computer, a heart sound and/or a lung sound based on a measurement position of a probe for measuring a heart sound and/or a lung sound of a patient;
analyzing, by the computer, the heart sound and/or the lung sound based on the measurement position and determining, by the computer, a target measurement position of the probe based on the analyzing result; and
providing, by the computer, the target measurement position of the probe.

15. The method of claim 14, wherein acquiring, by the computer, the heart sound and/or the lung sound includes:
determining, by the computer, at least one measurement position of the probe; and
acquiring, by the computer, the heart sound and/or the lung sound at each of the at least one determined measurement position.

16. The method of claim 15, wherein determining, by the computer, the target measurement position of the probe includes:
extracting, by the computer, a signal waveform of the heart sound and/or the lung sound acquired at each of the at least one measurement position;
analyzing, by the computer, the signal waveform of the heart sound and/or the lung sound and determining, by the computer, whether the analyzed signal waveform is an appropriate signal waveform; and
deriving, by the computer, a measurement position of the probe corresponding to the appropriate signal waveform based on the determination result and determining, by the computer, the derived measurement position as the target measurement position.

17. The method of claim 14, wherein acquiring, by the computer, the heart sound and/or the lung sound includes:
filtering the heart sound and the lung sound based on a difference between frequency bands of the heart sound and/or the lung sound, and separating the heart sound and the lung sound from each other; or
separating the heart sound into a first heart sound and a second heart sound, based on an amplitude or an amplitude variation of a signal waveform of the heart sound and/or the lung sound; or
separating the lung sound into a left lung sound and a right lung sound, based on a frequency band of the heart sound and/or the lung sound.

18. The method of claim 17, wherein acquiring, by the computer, the heart sound and/or the lung sound includes:
acquiring the heart sound and/or the lung sound using a microphone, while a position of the microphone is adjusted based on percentages of the heart sound and lung sound to be acquired.

19. The method of claim 14, wherein the method further comprises:
analyzing, by the computer, the heart sound and/or the lung sound and determining, by the computer, whether an abnormal region is contained therein, based on the analysis result; and
when the computer determines that the heart sound and/or the lung sound contains the abnormal region, correcting, by the computer, a signal waveform of the abnormal region based on a predefined normal signal waveform.

20. The method of claim 19, wherein determining, by the computer, whether the abnormal region is contained therein includes:
acquiring, by the computer, training data learned from a plurality of patients; and
determining, by the computer, whether the abnormal region is contained therein, based on the training data,
wherein the training data is created by collecting, by the computer, the heart sound and/or the lung sound containing an abnormal region from a plurality of patients and performing, by the computer, deep learning on the collected heart sound and/or lung sound.
